(19) United States
(12) Patent Application Publication
(10) Pub. No.: US 2001/0021371 A1
(43) Pub. Date: Sep. 13, 2001

(54) Improvements in or relating to cardiac imaging

(76) Inventors: Morten Eriksen, Olso (NO); Jonny Ostensen, Oslo (NO); Sigmund Frigstad, Trondhaim (NO)

Correspondence Address: BACON & THOMAS, PLLC, 4th Floor, 625 Slaters Lane, Alexandria, VA, 22314-1176, US

(21) Appl. No.: 09/742,439

(22) Filed: Dec. 22, 2000

Related U.S. Application Data
(63) Continuation of application No. PCT/GB99/01966, filed on Jun. 23, 1999.
(60) Provisional application No. 60/092,483, filed on Jul. 10, 1998.

(30) Foreign Application Priority Data
Jun. 23, 1998 (GB) 9813588.4

(51) Int. Cl. A61K 49/22

(52) U.S. Cl. 424/9.52

(57) ABSTRACT

A method of assessing relative rates of blood flow in the coronary arteries which involves observing one or more flow parameters in respect of contrast agent-containing blood flowing in at least one coronary artery of a subject, by generating a sequence of ultrasound images of the heart in a plane at least substantially perpendicular to the cardiac axis. Ultrasound imaging techniques which may be employed include power Doppler imaging and second harmonic B-mode or power Doppler imaging.

This invention relates to a method of assessing relative rates of blood flow in the coronary arteries of a subject, more particularly to such a method using contrast agent-enhanced ultrasound imaging.

In many countries of the world coronary artery disease is the largest single cause of morbidity and death in middle aged people. It may occur through chronic development of a coronary artery stenosis or through sudden coronary artery occlusion; a chronic development usually leads to symptoms of chest pain, dyspnea or fatigue at subnormal levels of exercise, whereas an acute development may lead to acute chest pain and acute myocardial infarction.

At present, anatomical evaluation of disease processes involving the coronary arteries may only be performed by means of coronary arteriography, although a range of techniques are used to evaluate functional implications of such disease. Amongst the more commonly used of such techniques are exercise electrocardiograms, exercise or stress echocardiography and exercise or stress radionuclide cardiac imaging. Electrocardiograms are invariably used in the evaluation of acute chest pain, but imaging techniques such as echocardiography and radionuclide imaging are much less commonly used, in part because of their relatively low sensitivity and, in the case of radionuclide imaging, because of the limited availability and the high cost of appropriate facilities. Use of coronary arteriography is generally restricted to the acute phase in cases where there are clear indications supporting invasive reperfusion. Whilst conventional echocardiography techniques may be used to distinguish between completely unsuccessful reperfusion and partial or complete reperfusion, they have been found to be insufficiently sensitive to distinguish between partial flow and unimpeded flow; it will be appreciated that such information is a desirable aid to patient prognosis and management.

There is accordingly a need for methods which permit better evaluation of coronary artery disease, particularly in the acute phase, in cases where indications for coronary arteriography are not present, and/or in cases where electrocardiograms or other tests are inconclusive.

The present invention is based on the finding that contrast agent-enhanced ultrasound imaging of the coronary arteries may be used to assess relative rates of blood flow within those arteries. This facilitates the identification of any coronary artery affected by a stenosis, since such an artery will tend to exhibit a lower flow rate than a healthy artery; it may also be possible to assess the severity of any stenosis from the magnitude of the reduction in flow rate. The method of the invention is performed using intravenously injected contrast agent and may therefore be performed without cardiac catheterisation. Accordingly it provides a valuable non-invasive technique permitting selection of patients for coronary arteriography and/or rescue procedures such as percutaneous transthoracic cardioangioplasty to be made before catheterisation. The method may, for example, be used in the assessment of chronic coronary artery disease in subjects at rest or during physical or pharmacological stress, and in acute cases at rest so as to evaluate disease levels and/or potential for thrombolytic treatment.

There has been a number of disclosures relating to use of contrast agent-enhanced echocardiography in assessing cardiac perfusion. Such methods, however, typically involve imaging of the microvasculature of the myocardium. Thus, for example, WO-A-9817324 discloses contrast agents capable of temporary retention in tissue microvasculature; such agents are retained in, for example, myocardial tissue in concentrations proportional to the regional rate of tissue perfusion, so that ultrasound images in which the display is derived directly from return signal intensities (e.g. conventional or harmonic B-mode imaging) may be interpreted as perfusion maps in which the displayed signal intensity is a function of local perfusion.

Observation of the flow of contrast agent-containing blood in the coronary arteries in accordance with the present invention, on the other hand, has the advantage that significantly enhanced contrast effects may be observed, since the echogenicity of the contrast agent is not diluted by the low volume fraction of the microvasculature of the myocardium. Moreover, imaging of the coronary arteries per se may facilitate the use of Doppler-based imaging methods in view of the relatively high flow velocities of arterial blood.

A further advantage is that imaging of the coronary arteries permits well-defined estimates to be made in respect of flow rates of contrast agent-containing blood, as described in greater detail hereinafter. Measurements of perfusion in the microvasculature of the myocardium, however, will inevitably tend to be less precise since the in-flow of contrast agent will typically be spread over several seconds.

Viewed from one aspect thereof the invention provides a method of assessing relative rates of blood flow in coronary arteries of a human or non-human animal subject which comprises generating a sequence of ultrasound images of the heart of said subject in a plane at least substantially perpendicular to the cardiac axis, intravenously administering an effective amount of an ultrasound contrast agent to said subject, and observing one or more flow parameters in respect of contrast agent-containing blood flowing in at least one coronary artery.

Viewed from other aspects the invention provides for use of an ultrasound contrast agent in the above-defined method and for use of ultrasound contrast-enhancing material in the preparation of an ultrasound contrast agent useful in the above-defined method.

As indicated above, imaging is performed in a plane at least substantially perpendicular to the cardiac axis, i.e. in a short axis view. The imaging plane is therefore substantially perpendicular to the dominant direction of the large coronary arteries, so that individual arteries may readily be selectively imaged. It will be appreciated that using such an imaging plane more than one coronary artery, e.g. all the coronary arteries, may be observed simultaneously in a single imaging procedure, so that direct comparisons may be made between stenotic and healthy arteries.

A variety of ultrasound imaging modalities may be used. These may, for example, be based on transmission/reception of one pulse for each scanline, e.g. as in fundamental B-mode, second harmonic B-mode or other frequency-weighted single pulse/echo imaging techniques; transmission/reception of two pulses for each scanline, e.g. as in pulse or phase inversion B-mode imaging techniques; two pulse methods wherein the pulses have the same or, more preferably opposite phase, or a phase difference and wherein the received radio frequency signals are added, subtracted or treated with more composite functions for scanline formation; transmission/reception of more than two pulses for each scanline, e.g. as in colour Doppler imaging, power Doppler imaging, colour velocity imaging, loss of correlation imaging or other multiple pulse transmission/reception methods which may be used to analyse echos from structures in relative motion or microbubbles which may change size or disappear on exposure to ultrasound.

The above-mentioned methods may be used at different acoustic output levels such as low power (mechanical index, MI, 0.2-0.4), medium power (MI 0.40-0.8) or high power (MI 0.8-1.6). They may be used at different frame rates, for example one frame per heartbeat, one frame for every second or higher number of heartbeats, two or more frames per heartbeat, or at fixed rates not synchronised to the cardiac cycle, e.g. in the range 0.1-20 Hz.

Power Doppler imaging involves displaying the intensity of Doppler-shifted signals, and thereby permits selective imaging of movement in an imaged organ. Thus only ultrasound echo intensities from tissues or fluids moving at velocities above a certain threshold are coded and displayed, the velocity information contained in the return signal being discarded. Whilst existing power Doppler echocardiographic techniques may permit separation of signals in respect of blood flowing in the coronary arteries and heart chambers from background tissue echoes, for example by careful selection of instrument parameters such as wall filter settings and pulse repetition frequencies, the backscatter intensity from blood itself is often too low to be displayed, in part because of the overlapping ranges of blood and tissue velocities in the heart. The use of contrast agents in accordance with the method of the invention, however, substantially enhances backscatter from blood, e.g. more than 100-fold, and so permits the power Doppler display of images derived from blood in motion, even in relatively small arteries.

As is now well known, harmonic imaging techniques are of particular value in delineating contrast from resonant contrast agent moieties such as gas microbubbles as compared to contrast from relatively non-resonant tissue. Such techniques are therefore also particularly useful in the method of the invention. The use of second harmonic power Doppler imaging may be especially advantageous in terms of enhanced contrast specificity and low contrast agent dosage requirements.

It is also known that harmonic and/or power Doppler imaging techniques which use relatively high ultrasound energy inputs may induce destruction of at least part of the administered contrast agent, especially when gas-microbubble-containing contrast agents (e.g. as described in greater detail hereinafter) are employed. Such destruction events may in themselves generate “signature” signals capable of detection by the imaging equipment, for example apparent Doppler shifts such as the “acoustically stimulated acoustic emissions” described in WO-A-9325241. The observation of such signals may be advantageous in reducing myocardial contrast effects which may otherwise tend to obscure some of the coronary arteries.

A variety of flow parameters in respect of coronary arterial blood may be observed in accordance with the invention. Thus, for example, one may measure the time lapse occurring between appearance of contrast agent-induced signals in the left ventricle and the appearance of such signals in the coronary arteries. It will be appreciated that if a coronary artery has reduced flow due to a stenosis, then the time lapse before appearance of contrast agent-induced effects in this artery will be greater than for normal arteries; the magnitude of the time lapse difference will give an indication of the severity of the stenosis.

The mean transit time of contrast agent-induced effects in a particular artery will also give an indication of coronary flow, and may therefore be used as an alternative to time of appearance measurements.

One may also use Doppler imaging techniques in order directly to evaluate blood flow in coronary arteries.

Whilst time of appearance measurements in particular ideally require that the contrast agent should arrive in the left ventricle and coronary arteries with a sharper bolus front than will normally occur following intravenous injection, the action of the aortic valve and mixing effects in the left ventricle will in practice tend to create a stepwise rising bolus front permitting effective measurements to be made. If desired, a sharper contrast agent front may be formed by using high energy ultrasound to destroy contrast agent in the aortic root so as to generate a “negative” bolus which will be followed by a sharp front of “fresh” contrast agent. Such destruction may, for example, be achieved by application of intense continuous low frequency ultrasound irradiation, e.g. for 1-5 seconds.

Contrast agents capable of surviving several passages of circulation, for example stabilised gas microbubble-containing contrast agents such as those disclosed in WO-A-9729783, may be obtained in recirculating steady state concentrations following administration in a sufficient amount. Imaging procedures involving observation of contrast agents in such a “recirculating phase”, as well as contrast agents useful in such procedures, are described in WO-A-9908714.

In accordance with a further aspect of the present invention a subject previously administered with an effective ultrasound contrast agent such that said agent is uniformly distributed in the recirculating phase of the blood pool, may be subjected to ultrasound emission, e.g. from a scanner directed at the aortic root or the left ventricle, in order to destroy or discernibly modify the circulating contrast agent. Abrupt termination of the ultrasound emission will give a substantially sharp bolus front as further contrast agent is washed in, and this may be used for assessment of the rate of reappearance of contrast agent in the coronary arteries.

It will be appreciated that the imaging frame rate in imaging procedures in accordance with the invention should be as high as possible in order to determine appearance time delays, transit times etc. as accurately as possible. Time measurements may, for example, be made by frame counting or by establishing a region of interest around the major coronary arteries and performing a time intensity analysis.

As noted above, the subject may be subjected to stress, e.g. physical exercise or pharmacological stress, during imaging in accordance with the method of the invention. This may be advantageous in that in the case of a moderate stenosis blood flow in the affected coronary artery may tend to appear normal at rest as a result of autoregulation. During stress, however, blood flow in healthy coronary arteries will typically increase to 4-6 times its normal value, whereas flow in a stenotic artery will remain substantially unchanged because of exhaustion of the flow reserve. The distinction between normal and stenotic coronary arteries will therefore be substantially increased and the sensitivity of the method will be correspondingly enhanced.

Vasodilators are a preferred category of vasoactive substances which may be administered to induce pharmacological stress. Representative examples of vasodilator drugs which may be used in accordance with this embodiment of the method of the invention include adenosine, dipyridamole, nitroglycerine, isosorbide mononitrate, prazosin, doxazosin, hydralazine, dihydralazine, sodium nitroprusside, pentoxyphylline, amelodipine, felodipine, isradipine, nifedipine, nimodipine, verapamil, diltiazem and nitric oxide. Stress-inducing agents such as arbutamine and dobutamine, which have a secondary vasodilatation-inducing effect as a result of their metabolism-increasing effects, may similarly be used. Use of adenosine is particularly preferred since it is an endogenous substance and has a rapid but short-lived vasodilatating effect. This latter property is confirmed by the fact that it has a blood pool half-life of only a few seconds; possible discomfort to patients during vasodilatation is therefore minimised. Vasodilatation induced by adenosine will be most intense in the heart since the drug will tend to reach more distal tissues in less than pharmacologically active concentrations; it is therefore the vasodilator drug of choice in this aspect of the method of the invention.

In principle any ultrasound contrast agent may be used in the method of the invention, subject only to the requirement that the size and stability of the contrast agent moieties are such that they are capable, following intravenous injection, of passing through the lung capillaries and generating responses in the left ventricle of the heart and the coronary arteries. Contrast agents which comprise or are capable of generating gas microbubbles are preferred since microbubble dispersions, if appropriately stabilised, are particularly efficient backscatterers of ultrasound by virtue of the low density and ease of compressibility of the microbubbles.

Gases which may be used include any biocompatible substances, including mixtures, which are at least partially, e.g. substantially or completely, in gaseous or vapour form at the normal human body temperature of 37° C. Representative gases thus include air; nitrogen; oxygen; carbon dioxide; hydrogen; inert gases such as helium, argon, xenon or krypton; sulphur fluorides such as sulphur hexafluoride, disulphur decafluoride or trifluoromethylsulphur pentafluoride; selenium hexafluoride; optionally halogenated silanes such as methylsilane or dimethylsilane; low molecular weight hydrocarbons (e.g. containing up to 7 carbon atoms), for example alkanes such as methane, ethane, a propane, a butane or a pentane, cycloalkanes such as cyclopropane, cyclobutane or cyclopentane, alkenes such as ethylene, propene, propadiene or a butene, and alkynes such as acetylene or propyne; ethers such as dimethyl ether; ketones; esters; halogenated low molecular weight hydrocarbons (e.g. containing up to 7 carbon atoms); and mixtures of any of the foregoing. Advantageously at least some of the halogen atoms in halogenated gases are fluorine atoms; thus biocompatible halogenated hydrocarbon gases may, for example, be selected from bromochlorodifluoromethane, chlorodifluoromethane, dichlorodifluoromethane, bromotrifluoromethane, chlorotrifluoromethane, chloropentafluoroethane, dichlorotetrafluoroethane, chlorotrifluoroethylene, fluoroethylene, ethyl fluoride, 1,1-difluoroethane and perfluorocarbons. Representative perfluorocarbons include perfluoroalkanes such as perfluoromethane, perfluoroethane, perfluoropropanes, perfluorobutanes (e.g. perfluoro-n-butane, optionally in admixture with other isomers such as perfluoro-iso-butane), perfluoropentanes, perfluorohexanes or perfluoroheptanes; perfluoroalkenes such as perfluoropropene, perfluorobutenes (e.g. perfluorobut-2-ene), perfluorobutadiene, perfluoropentenes (e.g. perfluoropent-1-ene) or perfluoro-4-methylpent-2-ene; perfluoroalkynes such as perfluorobut-2-yne; and perfluorocycloalkanes such as perfluorocyclobutane, perfluoromethylcyclobutane, perfluorodimethylcyclobutanes, perfluorotrimethyl-cyclobutanes, perfluorocyclopentane, perfluoromethyl-cyclopentane, perfluorodimethylcyclopentanes, perfluorocyclohexane, perfluoromethylcyclohexane or perfluorocycloheptane. Other halogenated gases include methyl chloride, fluorinated (e.g. perfluorinated) ketones such as perfluoroacetone and fluorinated (e.g. perfluorinated) ethers such as perfluorodiethyl ether. The use of perfluorinated gases, for example sulphur hexafluoride and perfluorocarbons such as perfluoropropane, perfluorobutanes, perfluoropentanes and perfluorohexanes, may be particularly advantageous in view of the recognised high stability in the blood stream of microbubbles containing such gases. Other gases with physicochemical characteristics which cause them to form highly stable microbubbles in the blood stream may likewise be useful.

Representative examples of contrast agent formulations include microbubbles of gas stabilised (e.g. at least partially encapsulated) by a coalescence-resistant surface membrane (for example gelatin, e.g. as described in WO-A-8002365), a filmogenic protein (for example an albumin such as human serum albumin, e.g. as described in U.S. Pat. No. 4,718,433, U.S. Pat. No. 4,774,958, U.S. Pat. No. 4,844,882, EP-A-0359246, WO-A-9112823, WO-A-9205806, WO-A-9217213, WO-A-9406477, WO-A-9501187 or WO-A-9638180), a polymer material (for example a synthetic biodegradable polymer as described in EP-A-0398935, an elastic interfacial synthetic polymer membrane as described in EP-A-0458745, a microparticulate biodegradable polyaldehyde as described in EP-A-0441468, a microparticulate N-dicarboxylic acid derivative of a polyamino acid-polycyclic imide as described in EP-A-0458079, or a biodegradable polymer as described in WO-A-9317718 or WO-A-9607434), a non-polymeric and non-polymerisable wall-forming material (for example as described in WO-A-9521631), or a surfactant (for example a polyoxyethylene-polyoxypropylene block copolymer surfactant such as a Pluronic, a polymer surfactant as described in WO-A-9506518, or a film-forming surfactant such as a phospholipid, e.g. as described in WO-A-9211873, WO-A-9217212, WO-A-9222247, WO-A-9409829, WO-A-9428780, WO-A-9503835 or WO-A-9729783). Contrast agent formulations comprising free microbubbles of selected gases, e.g. as described in WO-A-9305819, or comprising a liquid-in-liquid emulsion in which the boiling point of the dispersed phase is below the body temperature of the subject to be imaged, e.g. as described in WO-A-9416739, may also be used.

Other useful gas-containing contrast agent formulations include gas-containing solid systems, for example microparticles (especially aggregates of microparticles) having gas contained therewithin or otherwise associated therewith (for example being adsorbed on the surface thereof and/or contained within voids, cavities or pores therein, e.g. as described in EP-A-0122624, EP-A-0123235, EP-A-0365467, WO-A-9221382, WO-A-9300930, WO-A-9313802, WO-A-9313808 or WO-A-9313809). It will be appreciated that the echogenicity of such microparticulate contrast agents may derive directly from the contained/associated gas and/or from gas (e.g. microbubbles) liberated from the solid material (e.g. upon dissolution of the microparticulate structure).

The disclosures of all of the above-described documents relating to gas-containing contrast agent formulations are incorporated herein by reference.

Gas microbubbles and other gas-containing materials such as microparticles preferably have an initial average size not exceeding 10 μm (e.g. of 7 μm or less) in order to permit their free passage through the pulmonary system. However, larger microbubbles may be employed where, for example, these contain a mixture of one or more relatively blood-soluble or otherwise diffusible gases such as air, oxygen, nitrogen or carbon dioxide with one or more substantially insoluble and non-diffusible gases such as perfluorocarbons. Outward diffusion of the soluble/diffusible gas content following administration will cause such microbubbles rapidly to shrink to a size which will be determined by the amount of insoluble/non-diffusible gas present and which may be selected to permit passage of the resulting microbubbles through the lung capillaries of the pulmonary system.

Contrast agents which are capable of temporary retention in tissue microvasculature, e.g. as a result of phase change effects such as are described in WO-A-9416739, through coadministration of a dispersed gas and a diffusible component as described in WO-A-9817324, or through affinity towards normal or diseased endothelium may be employed, since such agents will exhibit essentially free-flowing behaviour during imaging in accordance with the invention in view of the relatively large size of the major coronary arteries.

Where phospholipid-containing contrast agent formulations are employed in accordance with the invention, e.g. in the form of phospholipid-stabilised gas microbubbles, representative examples of useful phospholipids include lecithins (i.e. phosphatidylcholines), for example natural lecithins such as egg yolk lecithin or soya bean lecithin, semisynthetic (e.g. partially or fully hydrogenated) lecithins and synthetic lecithins such as dimyristoylphosphatidylcholine, dipalmitoylphosphatidylcholine or distearoylphosphatidylcholine; phosphatidic acids; phosphatidylethanolamines; phosphatidylserines; phosphatidylglycerols; phosphatidylinositols; cardiolipins; sphingomyelins; fluorinated analogues of any of the foregoing; mixtures of any of the foregoing and mixtures with other lipids such as cholesterol. The use of phospholipids predominantly (e.g. at least 75%) comprising molecules individually bearing net overall charge, e.g. negative charge, for example as in naturally occurring (e.g. soya bean or egg yolk derived), semisynthetic (e.g. partially or fully hydrogenated) and synthetic phosphatidylserines, phosphatidylglycerols, phosphatidylinositols, phosphatidic acids and/or cardiolipins, for example as described in WO-A-9729783, may be particularly advantageous.

Representative examples of materials useful in gas-containing contrast agent microparticles include carbohydrates (for example hexoses such as glucose, fructose or galactose; disaccharides such as sucrose, lactose or maltose; pentoses such as arabinose, xylose or ribose; α-, β- and γ-cyclodextrins; polysaccharides such as starch, hydroxyethyl starch, amylose, amylopectin, glycogen, inulin, pulullan, dextran, carboxymethyl dextran, dextran phosphate, ketodextran, aminoethyldextran, alginates, chitin, chitosan, hyaluronic acid or heparin; and sugar alcohols, including alditols such as mannitol or sorbitol), inorganic salts (e.g. sodium chloride), organic salts (e.g. sodium citrate, sodium acetate or sodium tartrate), X-ray contrast agents (e.g. any of the commercially available carboxylic acid and non-ionic amide contrast agents typically containing at least one 2,4,6-triiodophenyl group having substituents such as carboxyl, carbamoyl, N-alkylcarbamoyl, N-hydroxyalkylcarbamoyl, acylamino, N-alkylacylamino or acylaminomethyl at the 3- and/or 5-positions, as in metrizoic acid, diatrizoic acid, iothalamic acid, ioxaglic acid, iohexol, iopentol, iopamidol, iodixanol, iopromide, metrizamide, iodipamide, meglumine iodipamide, meglumine acetrizoate and meglumine diatrizoate), polypeptides and proteins (e.g. gelatin or albumin such as human serum albumin), and mixtures of any of the foregoing.

The following non-limitative Examples serve to illustrate the invention.

Preparation 1

Stabilised Perfluorobutane Microbubble Dispersion

Hydrogenated phosphatidylserine (5 mg/ml in a 1% w/w solution of propylene glycol in purified water) and perfluorobutane gas were homogenised in-line at 7800 rpm and ca. 40° C. to yield a creamy-white dispersion. This dispersion was fractionated to substantially remove undersized microbubbles (<2 μm) and the volume of the dispersion was adjusted to the desired microbubble concentration by adding aqueous sucrose to give a sucrose concentration of 92 mg/ml. 2 ml portions of the resulting dispersion were filled into 10 ml flat-bottomed vials specially designed for lyophilisation and the contents were lyophilised to give a white porous cake. The lyophilisation chamber was then filled with perfluorobutane and the vials were sealed and stored. Prior to use, water was added to a vial and the contents were gently hand-shaken for several seconds to give a perfluorobutane microbubble dispersion; Coulter counter analysis shower that the concentration of microbubbles in the dispersion was 1.1% v/v and the median microbubble size was 2.7 μm.

### EXAMPLE 1

Imaging of Normal Heart Coronary Arteries (Open Chest Procedure)

A midline sternotomy was performed on an anaesthetised 20 kg mongrel dog and the heart was suspended in a pericardial cradle. A 30 mm silicone rubber ultrasound transmission standoff was placed in front of the heart and a P5-3 probe of an ATL HDI 3000 ultrasound scanner was applied to image a short axis cross section of the heart. The scanner was set to image in harmonic power Doppler mode at high output power and normal frame rate, a modality known to suppress signals originating from slowly moving contrast-generating particles within solid tissue. An intravenous bolus injection of 0.5 ml of a 1:10 dilution of a microbubble dispersion from Preparation 1 was then given and the time of appearance of contrast in the imaged coronary arteries was evaluated from the scanner cine-loop recording. Appearance was seen to be fairly simultaneous in all the arteries, occuring about one second after appearance of contrast in the left ventricular cavity.

### EXAMPLE 2

Imaging of a Stenosed Coronary Artery (Open Chest Procedure)

A midline sternotomy is performed on an anaesthetised 20 kg mongrel dog and the heart is suspended in a pericardial cradle. A 30 mm silicone rubber ultrasound transmission standoff is placed in front of the heart and a P5-3 probe of an ATL HDI 3000 ultrasound scanner is applied to image a short axis cross section of the heart. An occluding snare and a transit-time ultrasonic flowmeter transducer are applied to the left anterior descending coronary artery and the flow therein is reduced to 50% of its normal value. The procedure described in Example 1 is then repeated. Appearance of contrast effect in the terminal part of the occluded artery is delayed by approximately half a second compared to the other arteries in the same image.

### EXAMPLE 3

Creation of Sharp Bolus Waveform by Ultrasound Irradiation

In a modification of the procedure of Example 1, an additional defocused ultrasound transducer capable of emitting 5 W of continuous power at 1 MHz is aimed towards the valvular area in the aortic root, through a water-filled balloon positioned in the upper mediastinum, but is initially not switched on. A continuous intravenous infusion of a 1:10 dilution of microbubble dispersion from Preparation 1 is given at a rate of 1 ml/minute and the heart is imaged as in Example 1. Once a steady state contrast effect is observed, the aortic root transducer is switched on and then switched off 5 seconds later (acoustical interference between the instrument units has the effect that power Doppler observations cannot be made during this 5 second interval). Very sudden reappearance of contrast is seen simultaneously in all the coronary arteries 0.5 seconds after the aortic root transducer is switched off.

### EXAMPLE 4

Imaging of Stenosed Coronary Artery with Sharp Bolus Waveform

The procedure of Example 3 is repeated with occlusion of the left anterior descending coronary artery as described in Example 2. Reappearance of contrast effects in the stenosed artery is delayed by about half a second compared to the normal arteries; assessment of the delay is easier than in Example 2 as a result of the more well-defined rising phase of the bolus.

### EXAMPLE 5

Imagine of Normal Heart Coronary Arteries (Closed Chest Procedure)

A 24 kg mongrel dog was anaesthetized and placed in a left lateral decubitus position. The fur on the anterior and left side of the chest was shaved, and a venflon cannula was placed in a right forelimb vein for contrast agent injection. Ultrasound imaging of the heart was performed on an ATL HDI 5000 system with a Phased Array P4-2 transducer. The imaging view was a short axis plane corresponding to the middle segments of the left ventricular wall in order to allow detection of the arrival of contrast agent in all vascular segments at approximately equal distances from the aortic outlet.

Pulse inversion imaging was performed using a relatively low frame rate (9 Hz) and a moderately low acoustic output (corresponding to a mechanical index of 0.4).

An intravenous bolus of a 1% v/v suspension of microbubbles (72 μl of microbubbles, prepared as in Preparation 1) was injected over 1 second followed by an immediate flush of 4 ml isotonic saline. The first signs of contrast agent were detected in the right ventricular cavity. After about 4 seconds, contrast agent was detected in the left ventricular cavity. After another 2 seconds, contrast agent was detected in branches of the coronary arteries, imaged as bright spots and short white lines within the myocardium in the left ventricular wall. The appearance of contrast agent was fairly simultaneous in all vascular territories. The spatial resolution of the image was superior to that obtained in Example 1.

### EXAMPLE 6

Imagine of Normal Heart Coronary Arteries (Open Chest Procedure)

A midline sternotomy was performed on the dog of Example 5 and the heart was suspended in a pericardial cradle. A 30 mm silicone rubber ultrasound transmission standoff was placed between the transducer and the epicardium. Ultrasound imaging was performed on an ATL HDI 5000 system with a Phased Array P4-2 transducer. The imaging view was a short axis plane on a midpapillary level.

A pulse inversion imaging modality was selected using an increased frame rate (49 Hz) and a lower mechanical index (0.2) than in Example 5.

An intravenous bolus of a 1% vol/vol suspension of microbubbles (72 μl of microbubbles, prepared as in Preparation 1) was injected over 1 second followed by an immediate flush of 4 ml isotonic saline.

Almost simultaneous appearance of contrast agent in the territories of the three main coronary arteries was observed, imaged as bright spots and white lines within the myocardium. In some places, these vessel structures could be seen to connect to epicardial structures. Clear spatial resolution was detected through use of this imaging technique, and there were fewer bright flash artefacts than were observed in Example 5, as a result of the increased frame rate.

### EXAMPLE 7

Imaging of Coronary Artery Stenosis (Open Chest Procedure)

In the sternotomized dog of Example 6 is positioned a flowmeter and an adjustable occluder proximal on the left anterior descending artery (LAD). Baseline flow is measured, and an intravenous infusion of 15 μg/kg per min of dobutamine is administered. The heart rate and systolic pressure is observed to increase, and the flowmeter reveals a four-fold increase in flow.

When the flow has stabilised, the adjustable occluder is tightened until the flowmeter shows a flow value similar to the baseline flow before dobutamine administration.

A pulse inversion imaging modality is selected using a high frame rate (49 Hz) and a low mechanical index (0.2).

Contrast agent is injected as described in Example 6. It is now observed that the contrast agent appears much earlier in the lateral, inferior and posterior septal aspects of the left ventricular wall than in the anterior septal and anterior wall segments.

As the anterior aspects of the left ventricular wall is supplied by the LAD coronary artery, it is concluded that the delayed appearance of contrast in the microvasculature of this wall can be interpreted as a sign of a stenosis in the artery.

### EXAMPLE 8

Imaging Following Destruction of Preadministered Contrast Agent

The procedure of Example 3 is repeated except that the contrast agent is administered as a single intravenous bolus injection of 2.5 ml of a 1:10 dilution of a microbubble dispersion from Preparation 1. the aortic root transducer is switched on 30 seconds after the injection, when there is a near-constant concentration of contrast agent in the blood pool, and is switched off 5 seconds later. The subsequent imaging results are comparable to those described in Example 3.

### EXAMPLE 9

Imaging of a Stenosed Coronary Artery

The procedure of Example 5 is repeated on a sternotomised dog in which the left anterior descending coronary artery is occluded as described in Example 2. The imaging results are comparable to those described in Example 4.

1. A method of assessing relative rates of blood flow in coronary arteries of a human or non-human animal subject which comprises:
generating a sequence of ultrasound images of the heart of said subject in a plane at least substantially perpendicular to the cardiac axis;
intravenously administering to said subject an effective amount of an ultrasound contrast agent comprising a dispersion of echogenic gas microbubbles; and
observing one or more flow parameters in respect of contrast agent-containing blood flowing in at least one coronary artery.

2. A method as claimed in claim 1 wherein said sequence of ultrasound images is generated by power Doppler imaging or second harmonic B-mode imaging.

3. A method as claimed in claim 2 wherein said sequence of ultrasound images is generated by second harmonic power Doppler imaging.

4. A method as claimed in claim 2 or claim 3 wherein imaging is effected using an ultrasound energy input which is sufficiently high to induce destruction of at least part of the administered contrast agent.

5. A method as claimed in any of the preceding claims wherein said one or more flow parameters are observed by measuring time lapse occurring between appearance of contrast agent-induced signals in the left ventricle of the heart and the appearance of such signals in one or more of the coronary arteries.

6. A method as claimed in any of claims 1 to 4 wherein said one or more flow parameters are observed by measuring mean transit time of contrast agent-induced effects in one or more of the coronary arteries.

7. A method as claimed in any of claims 1 to 4 wherein said one or more flow parameters are observed by Doppler evaluation of blood flow in one or more of the coronary arteries.

8. A method as claimed in any of the preceding claims wherein flow parameters in respect of all of the coronary arteries are observed in a single imaging procedure.

9. A method as claimed in any of the preceding claims wherein transient application of high energy ultrasound is effected to destroy contrast agent in the aortic root of the heart so as to form a sharp front of inflowing contrast agent following cessation of said transient application.

10. A method as claimed in claim 9 wherein said transient application comprises irradiation with continuous low frequency ultrasound for 1-5 seconds.

11. A method as claimed in any of the preceding claims wherein said subject is subjected to physical exercise or pharmacological stress during said method.

12. A method as claimed in claim 11 wherein said pharmacological stress is induced by administration of a vasoactive substance.

13. A method as claimed in claim 12 wherein said vasoactive substance is a vasodilator drug.

14. A method as claimed in claim 13 wherein said vasodilator drug is adenosine.

15. A method as claimed in any of the preceding claims wherein the contrast agent comprises a biocompatible gas.

16. A method as claimed in claim 15 wherein said gas comprises a sulphur fluoride or a perfluorocarbon.

17. A method as claimed in either claim 15 or claim 16 wherein said gas is stabilised by at least one surfactant.

18. Use of an ultrasound contrast agent in a method as claimed in any of the preceding claims.

19. Use of ultrasound contrast-enhancing material in the preparation of an ultrasound contrast agent for use in a method as claimed in any of claims 1 to 17.

20. A method of assessing the relative rates of blood flow in coronary arteries of a human or non-human animal subject previously administered with an effective amount of an ultrasound contrast agent comprising a dispersion of echogenic gas microbubbles such that said contrast agent is uniformly distributed in the recirculating phase of the blood pool, said method comprising:
sonicating the aortic root or the left ventricle using high energy ultrasound to destroy or discernibly modify contrast agent therein so as to form a discernable front of inflowing contrast agent upon cessation of said sonication;
generating a sequence of ultrasound images in a plane at least substantially perpendicular to the cardiac axis of the heart of said subject; and
observing one or more flow parameters in respect of said inflowing contrast agent front in at least one coronary artery.
